SEQUENCE LISTING (8 sequences)

SEQ ID NO: 1 - RNA, 13 nt, Artificial Sequence
  Description of Artificial Sequence mammalian Kozak sequence
gccgccaccaugg

SEQ ID NO: 2 - RNA, 20 nt, Artificial Sequence
  Description of Artificial Sequence CpG RNA 1668
uccaugacguuccugaugcu

SEQ ID NO: 3 - RNA, 20 nt, Artificial Sequence
  Description of Artificial Sequence CpG RNA 1826
uccaugacguuccugacguu

SEQ ID NO: 4 - RNA, 20 nt, Artificial Sequence
  Description of Artificial Sequence oligoribonucleotide RNA 1982 corresponding to ODN DNA 1982
uccaggacuucucucagguu

SEQ ID NO: 5 - RNA, 15 nt, Artificial Sequence
  Description of Artificial Sequence stabilizing sequence of general formula (C/U)CCAN(x)CCC(U/A)Py(x)UC(C/U)CC (see description page 17), (Py(x)) is pyrimidine
nccancccnnucncc

SEQ ID NO: 6 - DNA, 20 bp, Artificial Sequence
  Description of Artificial Sequence CpG DNA 1668
tccatgacgttcctgatgct

SEQ ID NO: 7 - DNA, 20 bp, Artificial Sequence
  Description of Artificial Sequence CpG DNA 1826
tccatgacgttcctgacgtt

SEQ ID NO: 8 - DNA, 20 bp, Artificial Sequence
  Description of Artificial Sequence DNA 1982, has the same sequence as CpG DNA 1826, with the exception that the CpG motif has been removed by an exchange of C for G
tccaggacttctctcaggtt

(19) United States
(12) Patent Application Publication
(10) Pub. No.: US 2013/0273001 A1
(43) Pub. Date: Oct. 17, 2013

(54) IMMUNOSTIMULATION BY CHEMICALLY MODIFIED RNA

(71) Applicant: Curevac GMBH, Tubingen (DE)

(72) Inventors: Ingmar HOERR, Stuttgart (DE); Florlan Von Der Mulbe, Stuttgart (DE); Steve Pascolo, Zurlch (CH)

(21) Appl. No.: 13/850,024

(22) Filed: Mar. 25, 2013

Related U.S. Application Data
(63) Continuation of application No. 11/025,858, filed on Dec. 28, 2004, now abandoned.

(30) Foreign Application Priority Data
Jul. 3, 2002 (DE) 10229872.6

(51) Int. Cl. A61K 39/39

(52) U.S. Cl. CPC A61K 39/39
USPC 424/85.2; 424/184.1; 424/193.1; 424/198.1; 424/204.1; 424/234.1; 424/274.1; 424/269.1; 424/277.1

(57) ABSTRACT

The present invention relates to an immunostimulating agent comprising at least one chemically modified RNA. The invention furthermore relates to a vaccine which comprises at least one antigen in combination with the immunostimulating agent. The immunostimulating agent according to the invention and the vaccine according to the invention are employed in particular against infectious disease or cancer diseases.

The present application is a Continuation of co-pending PCT Application No. PCT/EP2003/007175, filed Jul. 3, 2003 which in turn, claims priority from German Application Serial No. 102 29 872.6, filed on Jul. 3, 2002. Applicants claim the benefits of 35 U.S.C. §120 as to the PCT application and priority under 35 U.S.C. §119 as to said German application, and the entire disclosures of both applications are incorporated herein by reference in their entireties.

The present invention relates to an immunostimulating agent comprising at least one chemically modified RNA. The invention furthermore relates to a vaccine which comprises at least one antigen in combination with the immunostimulating agent. The immunostimulating agent according to the invention and the vaccine according to the invention are employed in particular against infectious diseases or cancerous diseases.

RNA in the form of mRNA, tRNA and rRNA plays a central role in the expression of genetic information in the cell. However, it has furthermore been shown in some studies that RNA is also involved as such in the regulation of several processes, in particular in the mammalian organism. In this context, RNA can assume the role of communication messenger substance (Benner, FEBS Lett. 1988, 232: 225-228). Furthermore, an RNA has been discovered which has a high homology with a normal mRNA, but which is not translated but exercises a function in intracellular regulation (Brown et al., Cell 1992, 71: 527-542). Such RNA which has a regulatory action is characterized by an incomplete sequence of the ribosome binding site (Kozak sequence: GCCGCCACCAUGG, wherein AUG forms the start codon (cf. Kozak, Gene Expr. 1991, 1(2): 117-125)), in which it differs from (normal) mRNA. It has furthermore been demonstrated that this class of regulatory RNA also occurs in activated cells of the immune system, e.g. CD4⁺-T cells (Liu et al., Genomics 1997, 39: 171-184).

Both with conventional and with genetic vaccination, the problem frequently arises that only a low and therefore often inadequate immune response is caused in the organism to be treated or inoculated. So-called adjuvants, i.e. substances which can increase and/or can influence in a targeted manner an immune response towards an antigen, are therefore often added to vaccines. Adjuvants which have been known for a long time in the prior art are e.g. aluminium hydroxide, Freund's adjuvant etc. However, such adjuvants generate undesirable side effects, e.g. very painful irritation and inflammation at the site of administration. Furthermore, toxic side effects, in particular tissue necroses, are also observed. Finally, these known adjuvants have the effect of only an inadequate stimulation of the cellular immune response, since only B cells are activated.

It is moreover known of bacterial DNA that it has an immunostimulating action because of the presence of non-methylated CG motifs, and for this reason such CpG DNA has been proposed as an immunostimulating agent by itself and as an adjuvant for vaccines; cf. U.S. Pat. No. 5,663,153. This immunostimulating property of DNA can also be achieved by DNA oligonucleotides stabilized by phosphorothioate modification (U.S. Pat. No. 6,239,116). Finally, U.S. Pat. No. 6,406,705 discloses adjuvant compositions which comprise a synergistic combination of a CpG oligodeoxyribonucleotide and a non-nucleic acid adjuvant.

However, the use of DNA as an immunostimulating agent or as an adjuvant in vaccines is disadvantageous from several aspects. DNA is degraded only relatively slowly in the bloodstream, so that when immunostimulating DNA is used a formation of anti-DNA antibodies may occur, which has been confirmed in an animal model in mice (Gilkeson et al., J. Clin. Invest. 1995, 95: 1398-1402). The possible persistence of the DNA in the organism can thus lead to a hyperactivation of the immune system, which is known to result in splenomegaly in mice (Montheith et al., Anticancer Drug Res. 1997, 12(5): 421-432). Furthermore, DNA can interact with the host genome, in particular can cause mutations by integration into the host genome. Thus e.g. the DNA introduced may be inserted into an intact gene, which represents a mutation which impedes or even completely switches off functioning of the endogenous gene. By such integration events, on the one hand enzyme systems vital for the cell may be switched off, and on the other hand there is also the risk of transformation of the cell modified in this way into a degenerated state if a gene which is decisive for regulation of cell growth is modified by the integration of the endogenous DNA. A risk of cancer formation therefore cannot be ruled out when DNA is used as an immunostimulating agent.

Riedl et al. (J. Immunol. 2002, 168(10): 4951-4959) disclose that RNA bonded to an Arg-rich domain of the HBcAg nucleocapsid causes a Th1-mediated immune response against HbcAg. The Arg-rich domain of the nucleocapsid has a similarity to protamines and binds nucleic acids non-specifically.

The present invention is therefore based on the object of providing a novel system for improving immunostimulation generally and vaccination in particular, which causes a particularly efficient immune response in the patient to be treated or to be inoculated but avoids the disadvantages of known immunostimulants.

This object is solved by the embodiments of the present invention characterized in the claims.

In particular, the invention provides an immunostimulating agent comprising at least one RNA which has at least one chemical modification. Thus, the use of the chemically modified RNA for the preparation of an immunostimulating agent is also disclosed according to the present invention.

The present invention is based on the surprising finding that chemically modified RNA activates to a particularly high degree cells of the immune system (chiefly antigen-presenting cells, in particular dendritic cells (DC), and the defence cells, e.g. in the form of T cells) and in this way stimulates the immune system of an organism. In particular, the immunostimulating agent according to the invention, comprising the chemically modified RNA, leads to an increased release of immune-controlling cytokines, e.g. interleukins, such as IL-6, IL-12 etc. It is therefore possible e.g. to employ the immunostimulating agent of the present invention against infections or cancer diseases by injecting it e.g. into the infected organism or the tumour itself. Examples which may mentioned of cancer diseases which can be treated with the immunostimulating agent according to the invention are malignant melanoma, colon carcinoma, lymphomas, sarcomas, small cell pulmonary carcinomas, blastomas etc. The immunostimulating agent is furthermore advantageously employed against infectious diseases (e.g. viral infectious diseases, such as AIDS (HIV), hepatitis A, B or C, herpes, herpes zoster (chicken-pox), German measles (rubella virus), yellow fever, dengue etc. (flaviviruses), influenza (influenza viruses), haemorrhagic infectious diseases (Marburg or Ebola viruses), bacterial infectious diseases, such as Legionnaire's disease (Legionella), gastric ulcer (Helicobacter), cholera (Vibrio), E. coli infections, Staphylococci infections, Salmonella infections or Streptococci infections (tetanus), protozoological infectious diseases (malaria, sleeping sickness, leishmaniasis, toxoplasmosis, i.e. infections by Plasmodium, Trypanosoma, Leishmania and Toxoplasma, or fungal infections, which are caused e.g. by Cryptococcus neoformans, Histoplasma capsulatum, Coccidioides immitis, Blastomyces dermatitidis or Candida albicans).

The term “chemical modification” means that the RNA contained in the immunostimulant according to the invention is modified by replacement, insertion or removal of individual or several atoms or atomic groups compared with naturally occurring RNA species.

Preferably, the chemical modification is such that the RNA contains at least one analogue of naturally occurring nucleotides.

In a list which is in no way conclusive, examples which may be mentioned of nucleotide analogues which can be used according to the invention are phosphoroamidates, phosphorothioates, peptide nucleotides, methylphosphonates, 7-deazaguanosine, 5-methylcytosine and inosine. The preparation of such analogues is known to an expert e.g. from the U.S. Pat. No. 4,373,071, U.S. Pat. No. 4,401,796, U.S. Pat. No. 4,415,732, U.S. Pat. No. 4,458,066, U.S. Pat. No. 4,500,707, U.S. Pat. No. 4,668,777, U.S. Pat. No. 4,973,679, U.S. Pat. No. 5,047,524, U.S. Pat. No. 5,132,418, U.S. Pat. No. 5,153,319, U.S. Pat. No. 5,262,530 and U.S. Pat. No. 5,700,642. It is particularly preferable if the RNA consists of nucleotide analogues, e.g. the abovementioned analogues.

As further chemical modifications there may be mentioned, for example, the addition of a so-called “5′ cap” structure, i.e. a modified guanosine nucleotide, in particular m7G(5′)ppp (5′(A,G(5′)ppp(5′)A and G(5′)ppp(5′)G.

According to a further preferred embodiment of the present invention, the chemically modified RNA consists of relatively short RNA molecules which comprise e.g. about 2 to about 1,000 nucleotides, preferably about 8 to about 200 nucleotides, particularly preferably 15 to about 31 nucleotides.

According to the invention, the RNA contained in the immunostimulating agent can be single- or double-stranded. In particular, double-stranded RNA having a length of 21 nucleotides can also be employed in this context as interference RNA in order to specifically switch off genes, e.g. of tumour cells, and in this way to kill these cells in a targeted manner or in order to inactivate active genes therein which are to be held responsible for malignant degeneration (Elbashir et al., Nature 2001, 411, 494-498).

Specific examples of RNA species which can be employed according to the invention result if the RNA has one of the following sequences: 5′-UCCAUGACGUUCCUGAUGCU-3,5′-UCCAUGACGUUCCUGACGUU-3′ or 5′-UCCAGGACUUCUCLICAGGUU-3′. It is particularly preferable in this context if the RNA species are phosphorothioate-modified.

The immunostimulating agent according to the invention can optionally comprise the chemically modified RNA in combination with a pharmaceutically acceptable carrier and/or vehicle.

To further increase the immunogenicity, the immunostimulating agent according to the invention can comprise one or more adjuvants. In this context, a synergistic action of chemically modified RNA according to the invention and the adjuvant is preferably achieved in respect of the immunostimulation. “Adjuvant” in this context is to be understood as meaning any compound which promotes an immune response. Various mechanisms are possible in this respect, depending on the various types of adjuvants. For example, compounds which allow the maturation of the DC, e.g. lipopolysaccharides, TNF-α or CD40 ligand, form a first class of suitable adjuvants. Generally, any agent which influences the immune system of the type of a “danger signal” (LPS, GP96 etc.) or cytokines, such as GM-CFS, can be used as an adjuvant which enables an immune response to be intensified and/or influenced in a controlled manner. CpG oligonucleotides can optionally also be used in this context, although their side effects which occur under certain circumstances, as explained above, are to be considered. Because of the presence of the immunostimulating agent according to the invention comprising the chemically modified RNA as the primary immunostimulant, however, only a relatively small amount of CpG DNA is necessary (compared with immunostimulation with only CpG DNA), which is why a synergistic action of the immunostimulating agent according to the invention and CpG DNA in general leads to a positive evaluation of this combination. Particularly preferred adjuvants are cytokines, such as monokines, lymphokines, interleukins or chemokines, e.g. IL-1, IL-2, IL-3, IL-4, IL-5, IL-6, IL-7, IL-8, IL-9, IL-10, IL-12, INF-α, INF-γ, GM-CFS, LT-α or growth factors, e.g. hGH. Further known adjuvants are aluminium hydroxide, Freund's adjuvant and the stabilizing cationic peptides and polypeptides mentioned below, such as protamine, as well as cationic polysaccharides, in particular chitosan. Lipopeptides, such as Pam3Cys, are furthermore also particularly suitable for use as adjuvants in the immunostimulating agent of the present invention; cf. Deres et al., Nature 1989, 342: 561-564.

In addition to the direct use for starting an immune reaction, e.g. against a pathogenic germ or against a tumour, the immunostimulating agent can also advantageously be employed for intensifying the immune response against an antigen. The chemically modified RNA can therefore be used for the preparation of a vaccine in which it acts as an adjuvant which promotes the specific immune response against the particular antigen or the particular antigens.

As an other embodiment, the present invention thus also provides a vaccine comprising the immunostimulating agent defined above and at least one antigen.

In the case of “conventional” vaccination, the vaccine according to the invention or the vaccine to be prepared using the chemically modified RNA comprises the at least one antigen itself. An “antigen” is to be understood as meaning any structure which can cause the formation of antibodies and/or the activation of a cellular immune response. According to the invention, the terms “antigen” and “immunogen” are therefore used synonymously. Examples of antigens are peptides, polypeptides, that is to say also proteins, cells, cell extracts, polysaccharides, polysaccharide conjugates, lipids, glycolipids and carbohydrates. Possible antigens are e.g. tumour antigens and viral, bacterial, fungal and protozoological antigens. Surface antigens of tumour cells and surface antigens, in particular secreted forms, of viral, bacterial, fungal or protozoological pathogens are preferred in this context. The antigen can of course also be present in the vaccine according to the invention in the form of a hapten coupled to a suitable carrier. Suitable carriers are known to the expert and include e.g. human serum albumin (HSA), polyethylene glycols (PEG) etc. The hapten is coupled to the carrier by processes known in the prior art, e.g. in the case of a polypeptide carrier via an amide bond to a Lys residue.

In the case of genetic vaccination with the aid of the vaccine according to the invention or the genetic vaccine to be prepared using the chemically modified RNA, an immune response is stimulated by introduction of the genetic information for the at least one antigen (in this case thus a peptide or polypeptide antigen) in the form of a nucleic acid which codes for this antigen, in particular a DNA or an RNA (preferably an mRNA), into the organism or into the cell. The nucleic acid contained in the vaccine is translated into the antigen, i.e. the polypeptide or an antigenic peptide, respectively, coded by the nucleic acid is expressed, as a result of which an immune response directed against this antigen is stimulated. In the case of vaccination against a pathological germ, i.e. a virus, a bacterium or a protozoological germ, a surface antigen of such a germ is therefore preferably used for vaccination with the aid of the vaccine according to the invention comprising a nucleic acid which codes for the surface antigen. In the case of use as a genetic vaccine for treatment of cancer, the immune response is achieved by introduction of the genetic information for tumour antigens, in particular proteins which are expressed exclusively on cancer cells, by administering a vaccine according to the invention which comprises the nucleic acid which codes for such a cancer antigen. As a result, the cancer antigen(s) is or are expressed in the organism, which causes an immune response which is directed actively against the cancer cells.

The vaccines according to the invention may in particular be taken into consideration for treatment of cancer diseases. A tumour-specific surface antigen (TSSA) or a nucleic acid which codes for such an antigen is preferably used in this context. Thus, the vaccine according to the invention can be employed for treatment of the cancer diseases mentioned above in respect of the immunostimulating agent according to the invention. Specific examples of tumour antigens which can be used according to the invention in the vaccine are, inter alia, 707-AP, AFP, ART-4, BAGE, β-catenin/m, Bcr-abl, CAMEL, CAP-1, CASP-8, CDC27/m, CDK4/m, CEA, CT, Cyp-B, DAM, ELF2M, ETV6-AML1, G250, GAGE, GnT-V, Gp100, HAGE, HER-2/neu, HLA-A*0201-R170I, HPV-E7, HSP70-2M, HAST-2, hTERT (or hTRT), iCE, KIAA0205, LAGE, LDLR/FUT, MAGE, MART-1/Melan-A, MC1R, myosin/m, MUC1, MUM-1, -2, -3, NA88-A, NY-ESO-1, p190 minor bcr-abl, Pml/RARα, PRAME, PSA, PSM, RAGE, RU1 or RU2, SAGE, SART-1 or SART-3, TEL/AML1, TPI/m, TRP-1, TRP-2, TRP-2/INT2 and WT1.

The vaccine according to the invention is furthermore employed against infectious diseases, in particular the infections mentioned above in respect of the immunostimulating agent according to the invention. In the case of infectious diseases also, the corresponding surface antigens having the highest antigenic potential or a nucleic acid which codes for these are preferably used in the vaccine. In the case of the said antigens of pathogenic germs or organisms, in particular in the case of viral antigens, this is typically a secreted form of a surface antigen, Polyepitopes and nucleic acids which code for these, in particular mRNAs, are furthermore preferably employed according to the invention, these preferably being polyepitopes of the abovementioned antigens, in particular surface antigens of pathogenic germs or organisms or tumour cells, preferably secreted protein forms.

Furthermore, a nucleic acid which codes for at least one antigen and can be contained in the vaccine according to the invention can also contain, in addition to the section which codes for an antigenic peptide or polypeptide, at least one further functional section which codes e.g. for a cytokine which promotes the immune response, in particular those mentioned above from the aspect of the “adjuvant”.

As already mentioned, the nucleic acid which codes for at least one antigen can be DNA or RNA. For introduction of the genetic information for an antigen into a cell or an organism, a suitable vector which contains a section which codes for the particular antigen is in general necessary in the case of a DNA, vaccine according to the invention. Specific examples of such vectors which may be mentioned are the vectors of the series pVITRO, pVIVO, pVAC, pBOOST etc. (InvivoGen, San Diego, Calif., USA), which are described under the URL http//www.invivogen.com, the disclosure content of which in this respect is included in its full scope in the present invention.

In connection with DNA vaccines according to the invention, various methods can be mentioned for introduction of the DNA into cells, such as e.g. calcium phosphate transfection, polyprene transfection, protoplast fusion, electroporation, microinjection and lipofection, lipofection being particularly preferred.

In the case of a DNA vaccine, however, the use of DNA viruses as the DNA vehicle is preferred. Such viruses have the advantage that because of their infectious properties, a very high rate of transfection is to be achieved. The viruses used are genetically modified, so that no functional infectious particles are formed in the transfected cell.

From the aspect of safety, the use of RNA as the nucleic acid which codes for at least one antigen in the vaccine according to the invention is preferred. In particular, RNA does not bring with it the danger of becoming integrated in a stable manner into the genome of the transfected cell. Furthermore, no viral sequences, such as promoters, are necessary for effective transcription. RNA is moreover degraded considerably more easily in vivo. No anti-RNA antibodies have been detected to date in the blood circulation, evidently because of the relatively short half-life time of RNA compared with DNA.

It is therefore preferable according to the invention if the nucleic acid which codes for at least one antigen is an mRNA which contains a section which codes for at least one peptide antigen or at least one polypeptide antigen.

Compared with DNA, however, RNA is considerably more unstable in solution. RNA-degrading enzymes, so-called RNases (ribonucleases), are responsible for the instability. Even very small impurities of ribonucleases are sufficient to degrade RNA completely in solution. Such RNase impurities can generally be eliminated only by special treatments, in particular with diethyl pyrocarbonate (DEPC). The natural degradation of mRNA in the cytoplasm of cells is very precisely regulated. Several mechanisms are known in this respect. Thus, the terminal structure is of decisive importance for a functional mRNA. The so-called “cap structure” (a modified guanosine nucleotide) is to be found at the 5′ terminus, and a sequence of up to 200 adenosine nucleotides (the so-called poly-A tail) is to be found at the 3′ terminus. The RNA is recognized as mRNA and the degradation regulated via these structures. There are moreover further processes which stabilize or destabilize RNA. Many of these processes are still unknown, but an interaction between the RNA and proteins often seems to be decisive for this. For example, an mRNA surveillance system has recently been described (Hellerin and Parker, Ann. Rev. Genet. 1999, 33: 229 to 260), in which incomplete or nonsense mRNA is recognized by certain feedback protein interactions in the cytosol and rendered accessible to degradation, the majority of these processes being brought to completion by exonucleases.

It is therefore preferable to stabilize both the chemically modified RNA according to the invention and the RNA, in particular an mRNA, which is optionally present in the vaccine and codes for an antigen, against degradation by RNases.

The stabilization of the chemically modified RNA and, where appropriate, of the mRNA which codes for at least one antigen can be carried out by a procedure in which the chemically modified RNA or the mRNA which is optionally present and codes for the antigen is associated or complexed with or bonded linked to a cationic compound, in particular a polycationic compound, e.g. a (poly)cationic peptide or protein. In particular, the use of protamine as a polycationic nucleic acid-binding protein is particularly effective in this context. The use of other cationic peptides or proteins, such as poly-L-lysine or histones, is furthermore also possible. This procedure for stabilizing the modified mRNA is described in EP-A-1083232, the disclosure content of which in this respect is included in its full scope in the present invention. Further preferred cationic substances which can be used for stabilizing the chemically modified RNA and/or the mRNA optionally contained in the vaccine according to the invention include cationic polysaccharides, e.g. chitosan. The association or complexing with cationic compounds also improves the transfer of the RNA molecules into the cells to be treated or the organism to be treated.

In the sequences of eukaryotic mRNAs there are destabilizing sequence elements (DSE) which bind signal proteins and regulate enzymatic degradation of the mRNA in vivo. For further stabilization of the mRNA contained in the vaccine according to the invention, in particular in the region which codes for the at least one antigen, one or more modifications are therefore made compared with the corresponding region of the wild-type mRNA, so that it contains no destabilizing sequence elements. It is of course also preferable according to the invention to optionally eliminate from the mRNA any DSE present in the untranslated regions (3′- and/or 5′-UTR). In respect of the immunostimulating agent according to the invention, it is also preferable for the sequence of the chemically modified RNA contained therein to have no such destabilizing sequences.

Examples of the above DSE are AU-rich sequences (AURES), which occur in the 3′-UTR sections of numerous unstable mRNAs (Caput at al., Proc. Natl. Acad. Sci. USA 1986, 83: 1670 to 1674). The RNA molecules contained in the vaccine according to the invention are therefore preferably modified compared with the wild-type mRNA such that they have no such destabilizing sequences. This also applies to those sequence motifs which are possibly recognized by endonucleases, e.g. the sequence GAACAAG, which is contained in the 3′-UTR segment of the gene which codes for the transferrin receptor (Binder et al., EMBO J. 1994, 13: 1969 to 1980). Preferably, these sequence motifs are also eliminated from the chemically modified RNA molecules of the immunostimulating agent according to the invention or optionally from the mRNA present in the vaccine according to the invention.

The mRNA molecules which can be contained in the vaccine according to the invention also preferably have a 5′ cap structure. Examples of cap structures which may be mentioned are again m7G(5′)ppp (5′(A,G(5′)ppp(5′)A and G(5′)ppp(5′)G. The mRNA, as explained above in respect of the chemically modified RNA, can furthermore also contain analogues of naturally occurring nucleotides.

According to a further preferred embodiment of the present invention, the mRNA contains a polyA tail of at least 50 nucleotides, preferably at least 70 nucleotides, more preferably at least 100 nucleotides, particularly preferably at least 200 nucleotides.

For an efficient translation of the mRNA which codes for at least one antigen, effective binding of the ribosomes to the ribosome binding site (Kozak sequence: GCCGCCACCAUGG, the AUG forms the start codon) is furthermore necessary. In this respect, it has been found that an increased A/U content around this site renders possible a more efficient ribosome binding to the mRNA.

It is furthermore possible to insert one or more so-called IRES (internal ribosomal entry site) into the mRNA which codes for at least one antigen. An IRES can thus function as the sole ribosome binding site, but it can also serve to provide an mRNA which codes for several peptides or polypeptides which are to be translated by the ribosomes independently of one another (“multicistronic mRNA”). Examples of IRES sequences which can be used according to the invention are those from picornaviruses (e.g. FMDV), pestiviruses (CFFV), polioviruses (PV), encephalomyocarditis viruses (ECMV), foot and mouth disease viruses (FMDV), hepatitis C viruses (HCV), conventional swine fever viruses (CSFV), mouse leukoma virus (MLV), simian immunodeficiency viruses (SIV) or cricket paralysis viruses (CrPV).

According to a further preferred embodiment of the present invention, the mRNA has stabilizing sequences in the 5′ and/or 3′ untranslated regions which are capable of increasing the half-life time of the mRNA in the cytosol.

These stabilizing sequences can have a 100% sequence homology to naturally occurring sequences which occur in viruses, bacteria and eukaryotes, but can also be partly or completely synthetic in nature. The untranslated sequences (UTR) of the β-globin gene, e.g. from Homo sapiens or Xenopus laevis, may be mentioned as an example of stabilizing sequences which can be used in the present invention. Another example of a stabilizing sequence has the general formula (C/U)CCANₓCCC(U/A)PyₓUC(C/U)CC, which is contained in the 3′-UTR of the very stable mRNA which codes for α-globin, α-(I)-collagen, 15-lipoxygenase or for tyrosine hydroxylase (cf. Holcik et al., Proc. Natl. Acad. Sci. USA 1997, 94: 2410 to 2414). Such stabilizing sequences can of course be used individually or in combination with one another as well as in combination with other stabilizing sequences known to an expert.

To further increase the translation efficiency of the mRNA optionally contained in the vaccine according to the invention, the region which codes for the at least one antigen (and any further coding section optionally contained therein) can have the following modifications, compared with a corresponding wild-type mRNA, which can be present either alternatively or in combination.

On the one hand, the G/C content of the region of the modified mRNA which codes for the peptide or polypeptide can be greater than the G/C content of the coding region of the wild-type mRNA which codes for the peptide or polypeptide, the coded amino acid sequence being unchanged compared with the wild-type.

This modification is based on the fact that for efficient translation of an mRNA, the sequence (order) of the region of the mRNA to be translated is important. The composition and the sequence of the various nucleotides play a large role here. In particular, sequences having an increased G (guanosine)/C (cytosine) content are more stable than sequences having an increased A (adenosine)/U (uracil) content. According to the invention, the codons are therefore varied compared with the wild-type mRNA, while retaining the translated amino acid sequence, such that they contain an increased content of G/C nucleotides. Since several codons code for one and the same amino acid (degeneration of the genetic code), the codons which are most favourable for the stability can be determined (alternative codon usage).

Depending on the amino acid to be coded by the mRNA, various possibilities are possible for modification of the mRNA sequence compared with the wild-type sequence. In the case of amino acids which are coded by codons which contain exclusively G or C nucleotides, no modification of the codons is necessary. Thus, the codons for Pro (CCC or CCG), Arg (CCC or CGG), Ala (GCC or GGG) and Gly (GGG or GGG) require no change since no A or U is present.

In the following cases, the codons which contain A and/or U nucleotides are modified by substitution of other codons which code the same amino acids but contain no A and/or U. Examples are:

the codons for Pro can be modified from CCU or CCA to CCC or CCG;
the codons for Arg can be modified from CGU or CGA or AGA or AGG to GGG or CGG;
the codons for Ala can be modified from GCU or GCA to GCC or GGG;
the codons for Gly can be modified from GGU or GGA to GGC or GGG.

In other cases, A or U nucleotides indeed cannot be eliminated from the codons, but it is possible to reduce the A and U content by using codons which contain less A and/or U nucleotides. For example:

the codons for Phe can be modified from UUU to UUC;
the codons for Leu can be modified from LSA, CUU or CUA to CUC or CUG;
the codons for Ser can be modified from UCU or UCA or AGU to UCC, UCG or AGC;
the codon for Tyr can be modified from UAU to UAC;
the stop codon UAA can be modified to UAG or UGA;
the codon for Cys can be modified from UGU to UGC;
the codon for His can be modified from CAU to CAC;
the codon for Gln can be modified from CAA to CAG;
the codons for Ile can be modified from AUU or AUA to AUC;
the codons for Thr can be modified from ACU or ACA to ACC or ACG;
the codon for Asn can be modified from AAU to AAC;
the codon for Lys can be modified from AAA to AAG;
the codons for Val can be modified from GUU or GUA to GUC or GUG;
the codon for Asp can be modified from GAU to GAC;
the codon for Glu can be modified from GAA to GAG.

In the case of the codons for Met (AUG) and Trp (UGG), on the other hand, there is no possibility for modification of the sequence.

The abovementioned substitutions can of course be used individually or also in all possible combinations for increasing the G/C content of the modified mRNA compared with the original sequence. Thus, for example, all the codons for Thr occurring in the original (wild-type) sequence can be modified to ACC (or ACG). Preferably, however, combinations of the above substitution possibilities are used, e.g.:

substitution of all the codons, which code for Thr in the original sequence, to ACC (or ACG) and substitution of all the codons, which originally code for Ser, to UCC (or UCG or AGC);
substitution of all the codons, which code for Ile in the original sequence, to AUG and substitution of all the codons, which originally code for Lys, to AAG and substitution of all the codons, which originally code for Tyr, to UAC;
substitution of all the codons, which code for Val in the original sequence, to GUC (or GUG) and substitution of all the codons, which originally code for Glu, to GAG and substitution of all the codons, which originally code for Ala, to GCC (or GCG) and substitution of all the codons, which originally code for Arg, to CGC (or CGG);
substitution of all the codons, which code for Val in the original sequence, to GUC (or GUG) and substitution of all the codons, which originally code for Glu, to GAG and substitution of all the codons, which originally code for Ala, to GCC (or GCG) and substitution of all the codons, which originally code for Gly, to GGG (or GGG) and substitution of all the codons, which originally code for Asn, to AAC;
substitution of all the codons, which code for Val in the original sequence, to GUC (or GUG) and substitution of all the codons, which originally code for Phe, to UUC and substitution of all the codons, which originally code for Cys, to UGC and substitution of all the codons, which originally code for Leu, to CUG (or CUC) and substitution of all the codons, which originally code for Gln, to CAG and substitution of all the codons, which originally code for Pro, to CCC (or CCG);
etc.

Preferably, the G/C content of the region which codes for the antigenic peptide or polypeptide (or any other further section optionally present) in the mRNA is increased by at least 7%, more preferably by at least 15%, particularly preferably by at least 20% with respect to the G/C content of the coded region of the wild-type mRNA which codes for the corresponding peptide or polypeptide.

In this connection, it is particularly preferable to increase the G/C content of the mRNA modified in this way, in particular in the region which codes for the at least one antigenic peptide or polypeptide, to the maximum compared with the wild-type sequence.

A further preferred modification of an mRNA optionally contained in the vaccine characterized by the present invention is based on the finding that the translation efficiency is also determined by a different frequency in the occurrence of tRNAs in cells. If so-called “rare” codons are therefore present to an increased extent in an RNA sequence, the corresponding mRNA is translated significantly more poorly than in the case where codons which code for relatively “frequent” tRNAs are present.

Thus, according to the invention, the region which codes for the antigen (i.e. the peptide or polypeptide having an antigenic action) in the mRNA (which may be contained in the vaccine) is modified compared with the corresponding region of the wild-type mRNA such that at least one codon of the wild-type sequence which codes for a tRNA which is relatively rare in the cell is replaced by a codon which codes for a tRNA which is relatively frequent in the cell and which carries the same amino acid as the relatively rare tRNA.

By this modification, the RNA sequences are modified such that codons which are available for the frequently occurring tRNAs are inserted.

Which tRNAs occur relatively frequently in the cell and which, in contrast, are relatively rare is known to an expert; cf. e.g. Akashi, Curr, Opin. Genet. Dev. 2001, 11(6): 660-666.

According to the invention, by this modification all codons of the wild-type sequence which code for a tRNA which is relatively rare in the cell can in each case be exchanged for a codon which codes for a tRNA which is relatively frequent in the cell and which in each case carries the same amino acid as the relatively rare tRNA.

It is particularly preferable to combine the sequential G/C content which has been increased in the mRNA as described above, in particular to the maximum, with the “frequent” codons, without changing the amino acid sequence of the antigenic peptide or polypeptide (one or more) coded by the coding region of the mRNA. This preferred embodiment provides a particularly efficiently translated and stabilized mRNA for the vaccine according to the invention.

Preferably, the immunostimulating agent according to the invention comprises, in addition to the chemically modified RNA, and the vaccine according to the invention comprises, in addition to the immunostimulating agent, a pharmaceutically acceptable carrier and/or a pharmaceutically acceptable vehicle. Appropriate routes for suitable formulation and preparation of the immunostimulating agent according to the invention and the vaccine are disclosed in “Remington's Pharmaceutical Sciences” (Mack Pub. Co., Easton, Pa., 1980), the full content of which is a constituent of the disclosure of the present invention. Possible carrier substances for parenteral administration are e.g. sterile water, sterile sodium chloride solution, polyalkylene glycols, hydrogenated naphthalenes and, in particular, biocompatible lactide polymers, lactide/glycolide copolymers or polyoxyethylene/polyoxypropylene copolymers. Immunostimulating agents and vaccines according to the invention can comprise filler substances or substances such as lactose, mannitol, substances for covalent linking of polymers, such as e.g. of polyethylene glycol, on to antigenic haptens, peptides or polypeptides according to the invention, complexing with metal ions or inclusion of materials in or on particular preparations of polymer compounds, such as e.g. polylactate, polyglycolic acid, hydrogel or to liposomes, microemulsions, micelles, unilamellar or multilamellar vesicles, erythrocyte fragments or spheroblasts. The particular embodiments of the immunostimulating agent and the vaccine are chosen according to the physical properties, for example in respect of solubility, stability, bioavailability or degradability. Controlled or constant release of the active drug (-like) components according to the invention in the vaccine or in the immunostimulating agent includes formulations based on lipophilic depots (e.g. fatty acids, waxes or oils). In the context of the present invention, coatings of immunostimulating substances and vaccine substances or vaccine compositions (all of them according to the invention) comprising such substances, namely coatings with polymers, are also disclosed (e.g. polyoxamers or polyoxamines). Immunostimulating or vaccine substances or compositions according to the invention can furthermore have protective coatings, e.g. protease inhibitors or permeability intensifiers. Preferred carriers are typically aqueous carrier materials, water for injection (WFI) or water buffered with phosphate, citrate, HEPES or acetate etc. being used, and the pH is typically adjusted to 5.0 to 8.0, preferably 6.5 to 7.5. The carrier or the vehicle will additionally preferably comprise salt constituents, e.g. sodium chloride, potassium chloride or other components which render the solution e.g. isotonic. Furthermore, the carrier or the vehicle can contain, in addition to the abovementioned constituents, additional components, such as human serum albumin (HSA), polysorbate 80, sugars or amino acids.

The mode and method of administration and the dosage of the immunostimulating agent according to the invention and of the vaccine according to the invention depend on the nature of the disease to be cured, where appropriate the stage thereof, the antigen (in the case of the vaccine) and also the body weight, the age and the sex of the patient.

The concentration of the chemically modified RNA and also of the coding nucleic acid optionally contained in the vaccine in such formulations can therefore vary within a wide range from 1 μg to 100 mg/ml. The immunostimulating agent according to the invention and also the vaccine according to the invention are preferably administered to the patient parenterally, e.g. intravenously, intraarterially, subcutaneously or intramuscularly. It is also possible to administer the immunostimulating agent or the vaccine topically or orally.

The invention therefore also provides a method for the prevention and/or treatment of the abovementioned diseases which comprises administration of the immunostimulating agent according to the invention or the vaccine according to the invention to a patient, in particular to a human.

The figures show:

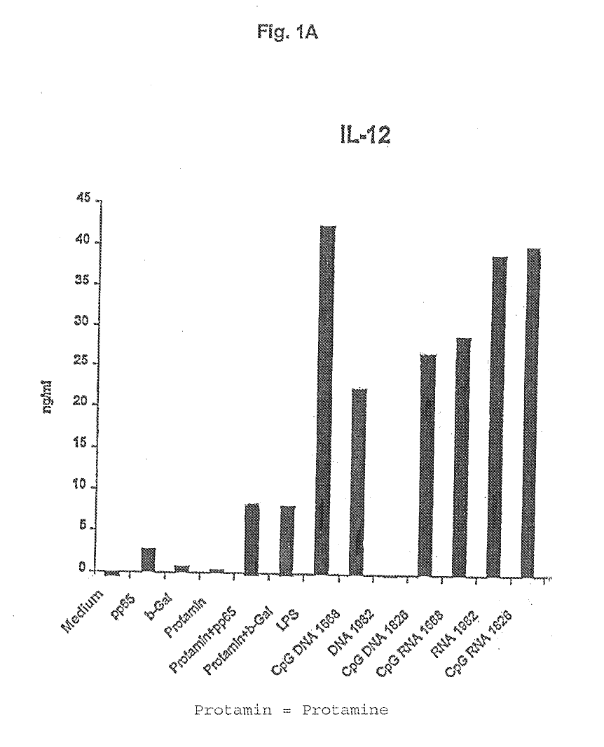

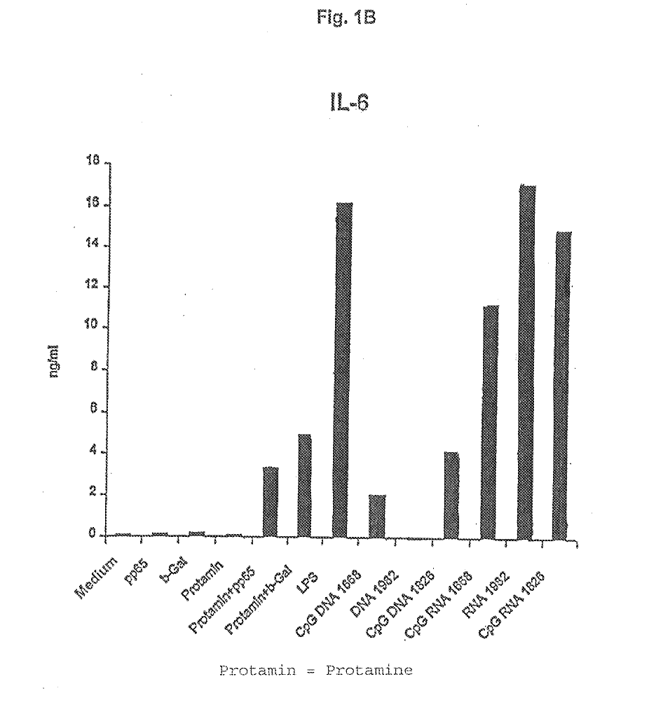

FIG. 1 shows results of stimulation of the maturation of dendritic cells (DC) of the mouse by chemically modified RNA according to the invention compared with mRNA, protamine-associated mRNA and DNA. DC of the mouse were stimulated with 10 μg/ml mRNA (pp65 for pp65 mRNA, β-Gal for β-galactosidase mRNA), mRNA stabilized by protamine (protamine+pp65, protamine+β-Gal), DNA (CpG DNA 1668, DNA 1982 and CpG DNA 1826) and phosphorothioate-modified RNA (RNA 1668, RNA 1982 and RNA 1826) and the DC activation was determined by measuring the release of IL-12 (FIG. 1A) and IL-6 (FIG. 1B) by means of cytokine ELISA. In each case medium without nucleic acid samples and medium with added protamine served as negative controls in the two series of experiments. Lipopolysaccharide (LPS) was used as a positive comparison. The oligodeoxyribonucleotides (ODN) CpG DNA 1668 and CpG DNA 1826 each contain a CpG motif. It is known of such ODN that they cause stimulation of DC (cf. U.S. Pat. No. 5,663,153). The ODN DNA 1982 has the same sequence as CpG DNA 1826, with the exception that the CpG motif has been removed by an exchange of C for G. The oligoribonucleotides CpG RNA 1668, RNA 1982 and CpG RNA 1826 according to the invention which have been stabilized by phosphorothioate modification correspond in their sequence to the abovementioned comparison ODN of the respective identification number. Compared with normal mRNA, the protamine-stabilized mRNA species show only a weak activation of the DC. A very much greater release of interleukin compared with this, however, is caused in both experiments by the phosphorothioate-modified oligoribonucleotides according to the invention, the values of which being comparable to those of the positive control (LPS). Compared with protamine-associated mRNA, a more than doubled release of IL-12 and IL-6 results on stimulation by phosphorothioate-modified oligoribonucleotides. This surprisingly high release of interleukin due to the oligoribonucleotides according to the invention is furthermore independent of CpG motifs, as shown by the comparison of the phosphorothioate-modified oligoribonucleotide RNA 1982 according to the invention with the corresponding ODN DNA 1982. The ODN DNA 1982 causes no release of interleukin in the DC, while RNA 1982 has the effect of release of interleukin, which in the case of IL-12 is comparable to that of the positive control LPS, and in the case of IL-6 even exceeds this.

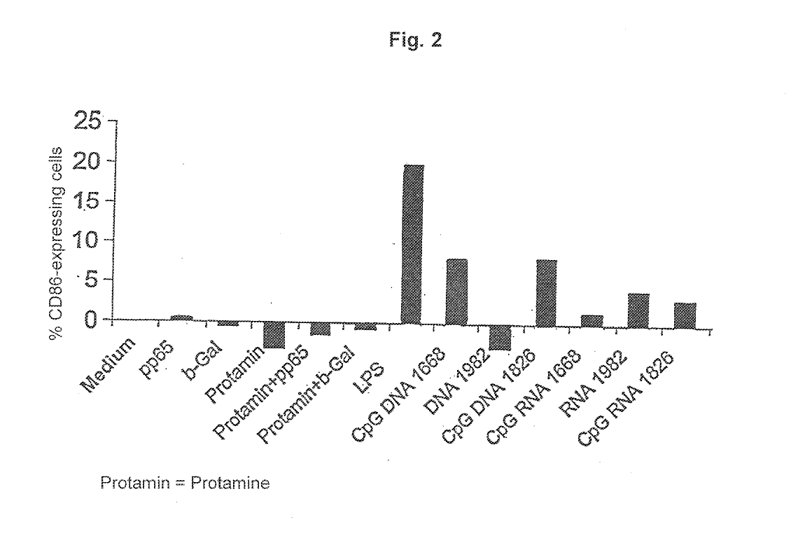

FIG. 2 shows the results of the determination of the expression of a surface activation marker (CD86) in DC which have been treated with the samples as described above for FIG. 1. For determination of the CD86 expression, some of the DC were labelled with an anti-CD86-specific monoclonal antibody 3 days after treatment of the DC with the samples described, and the percentage content of CD86-expressing cells was determined by means of flow cytometry. A significant CD86 expression is observed only in the comparison ODN, which have a CpG motif, and the phosphorothioate-modified RNA species according to the invention. However, all the values of the nucleic acid stimulants were significantly below the positive control (LPS). Furthermore, the CD86 determination confirms that the DC activation caused by phosphorothioate-modified RNA according to the invention is independent of CpG motifs, in contrast to DNA species: while the CpG-free ODN DNA 1982 causes no CD86 expression, in the case of the corresponding phosphorothioate-modified oligoribonucleotide RNA 1982, a CD86 expression is detected in 5% of the DC.

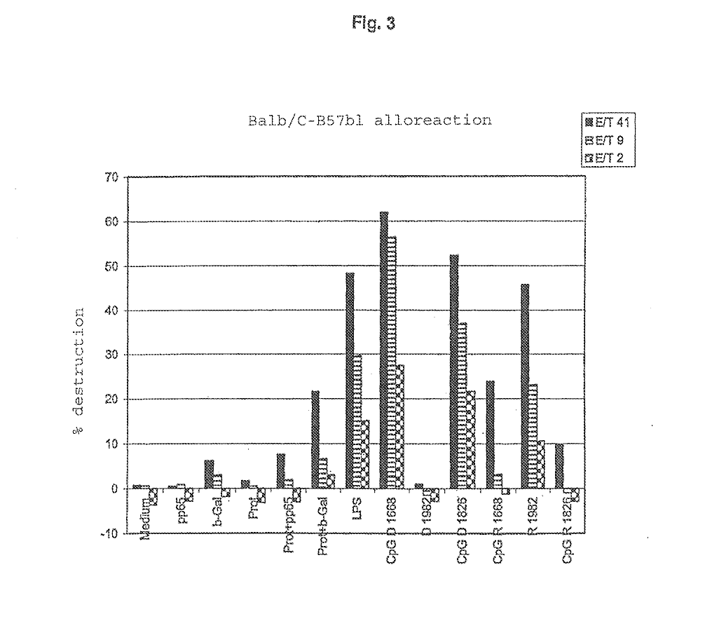

FIG. 3 shows the results of an alloreaction test using DC which were activated in vitro with the samples shown on the x axis (cf. also FIG. 1). 3 days after the stimulation, the DC were added to fresh spleen cells from an allogenic animal, and six days later were used in a cytotoxicity test in which the release of ⁵¹Cr was measured on target cells (P 815) compared with control cells (EL 4). The target and control cells were plated out in a constant amount and then incubated for 4 hours with in each case three different dilutions of the spleen cells co-cultured with DC (effector cells), so that a ratio of effector cells (E) to target cells (or control cells) (T) of 41:1, 9:1 and 2:1 resulted. The specific destruction in percent is stated on the y axis, and is calculated as follows: [(released radioactivity measured−spontaneously released radioactivity)/(maximum release of radioactivity−spontaneously released radioactivity)]×100. DC stimulated with protamine-associated β-galactosidase mRNA are capable of causing only a 20% specific destruction of target cells by the effector cells at the lowest dilution. In contrast, DC stimulated by phosphorothioate-modified oligoribonucleotide cause an almost 60%, that is to say about trebled, specific destruction of the target cells by the effector cells at the lowest dilution. This value is comparable to that of the positive control (LPS) and a comparison ODN containing a CpG motif (CpG DNA 1668). In contrast, an ODN without a CpG motif (DNA 1982) is inactive, which confirms the results from the preceding experiments according to FIG. 1 and FIG. 2. pp65 mRNA (without protamine), β-galactosidase mRNA (without protamine) and protamine and medium alone cause no specific destruction.

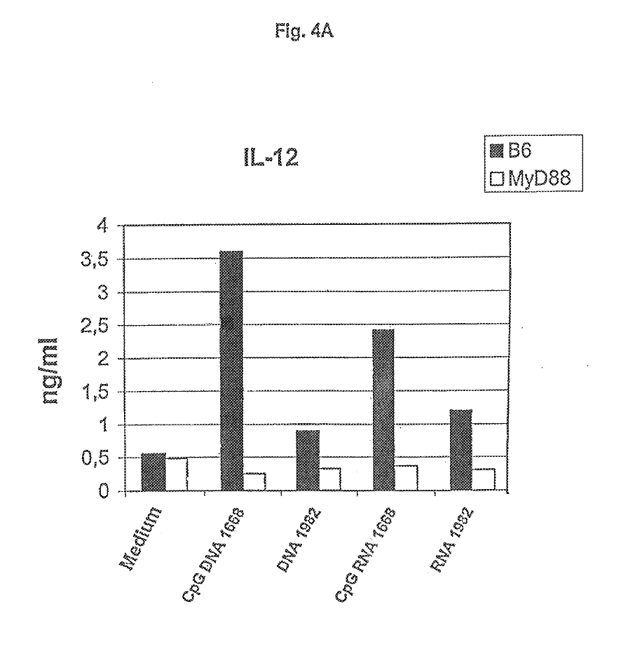

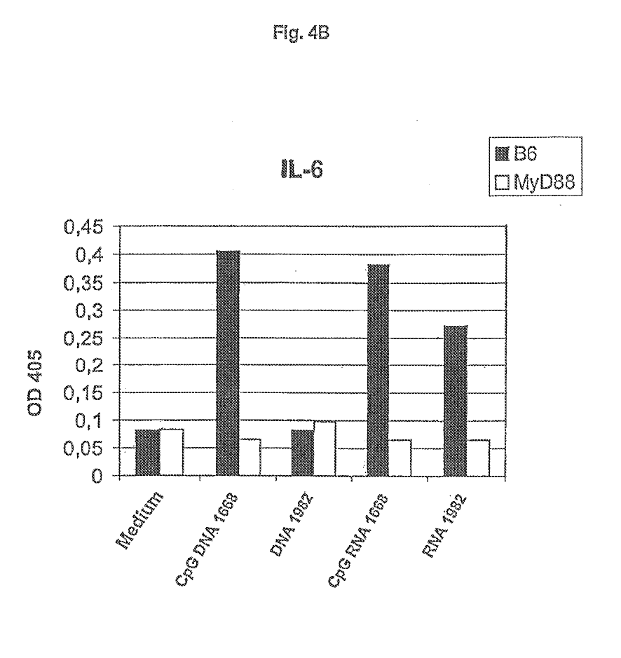

FIG. 4 shows results on the stimulation of maturation of dendritic cells (DC) from B6 mice, compared with MyD88 knock-out mice, by chemically modified oligoribonucleotides according to the invention and comparison. ODN. Stimulation only with medium served as a negative control. Stimulation took place as described before for FIG. 1 and the DC activation was determined by measuring the release of IL-12 (FIG. 4A) and IL-6 (FIG. 4B) by means of cytokine ELISA. In FIG. 4A, the IL-12 concentration is plotted in ng/ml on the y axis, while in FIG. 4B the absorption at 405 nm (absorption maximum of the detection reagent) is plotted on the y axis, this being directly proportional to the interleukin concentration. In MyD88 mice, the protein MyD88, a protein from the signal cascade of so-called toll-like receptors (TLR) is switched off. It is known from TLR-9 e.g. that it mediates activation of DC by CpG DNA. DC of B6 wild-type mice are activated by the phosphorothioate-modified oligoribonucleotides CpG RNA 1688 and RNA 1982 according to the invention and, as expected, by the comparison ODN CpG DNA 1668. The ODN DNA 1982 (without CpG motif) is again inactive. In contrast, none of the samples can bring about a noticeable release of IL-12 or IL-6 in DC from MyD88 mice. MyD88 therefore seems to be necessary for activation of DC by the chemically modified oligoribonucleotides according to the invention and by CpG ODN.

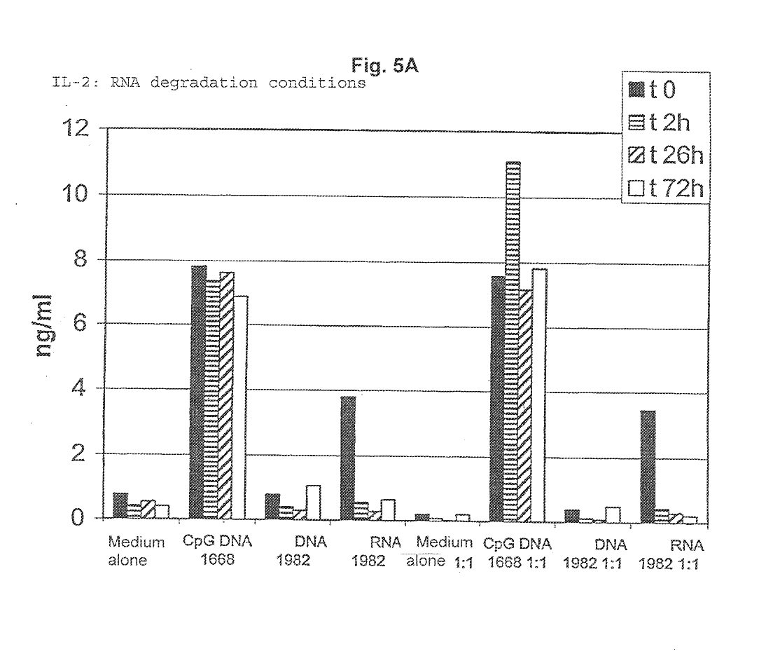

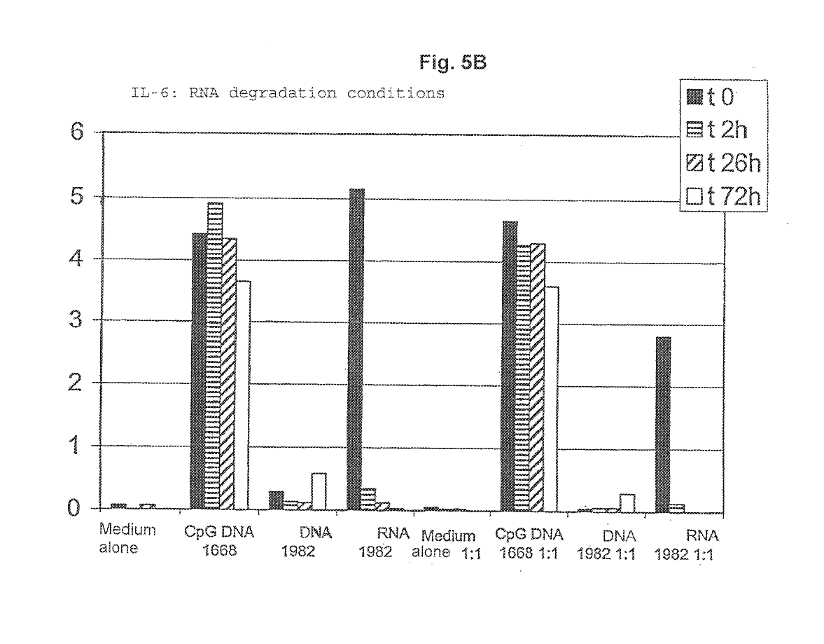

FIG. 5 shows results of the stimulation of DC by the chemically modified oligoribonucleotide RNA 1982 according to the invention and two comparison ODN which, before use for the DC activation, were incubated for 2, 26 or 72 h at 37° C. with medium which was not RNase-free. For comparison, in each case a sample was used without prior incubation (t=0). The samples identified with “1:1” were diluted 1:1 with buffer compared with the other particular samples. The DC activation was again measured by determination of the release of IL-12 (FIG. 5A) and IL-6 (FIG. 5B) by means of cytokine ELISA. The DC activation by CpG DNA is independent of a prior incubation with medium. As expected, the comparison ODN without a CpG motif leads to no release of interleukin. In the case of the oligoribonucleotide RNA 1982 according to the invention, a significant release of interleukin is measured without incubation with medium (t=0). Already after 2 h of incubation at 37° C. with medium which is not RNase-free, noticeable release of interleukin is no longer observed in the stimulation experiment with the oligoribonucleotide according to the invention.

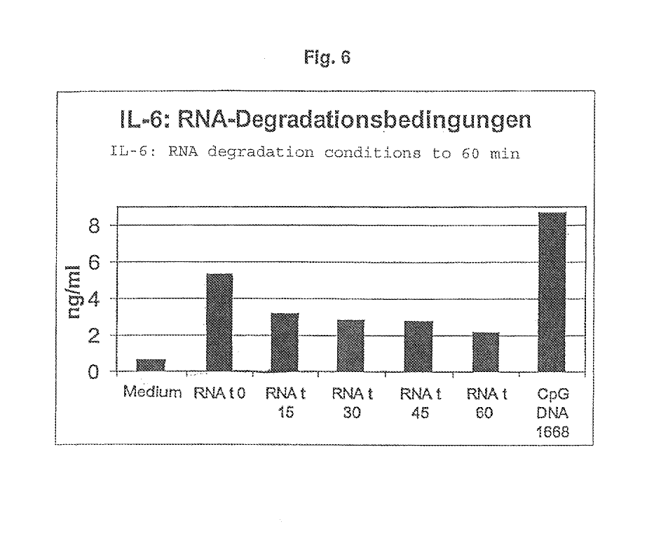

FIG. 6 shows the result of a similar experiment to that shown in FIG. 5B, but a more precise course with respect to time of the effect of the RNA degradation on the DC stimulation was recorded: The chemically modified oligoribonucleotide RNA 1982 according to the invention was again used for stimulation of DC and the activation of the DC was determined by measurement of the release of IL-6. Before the stimulation the oligoribonucleotide was incubated for 15, 30, 45 or 60 min with medium which was not RNase-free, as described above for FIG. 5. A sample which had not been incubated with the medium (t=0) again served as a comparison. The ODN CpG DNA 1668 was used as a positive control and medium alone was used as a negative control. Without prior incubation with medium which is not RNase-free, a potent DC activation by the chemically modified RNA according to the invention again results, as demonstrated by the IL-6 concentration of more than 5 ng/ml. This value falls to somewhat above 2 ng/ml within one hour of incubation under RNA degradation conditions. This shows that the chemically modified RNA is indeed degraded very much faster than DNA species under physiological, conditions, but the half-life is evidently sufficiently long for the immunostimulating action according to the invention to be displayed.

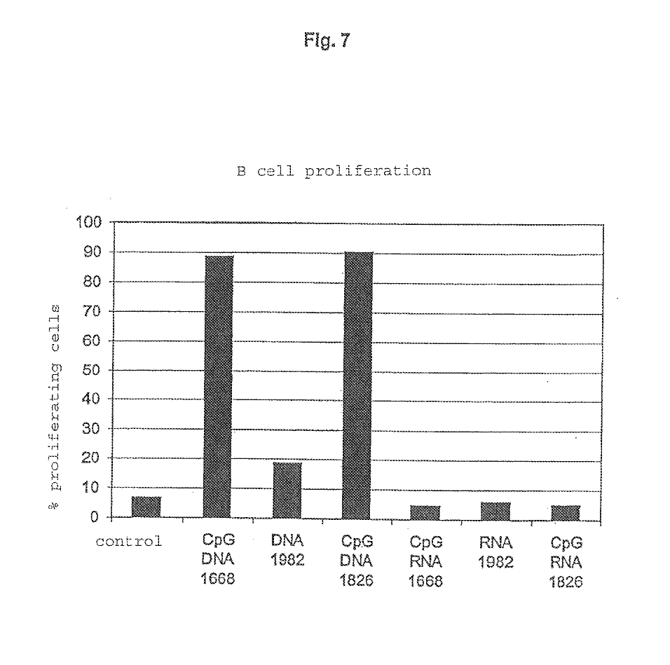

FIG. 7 shows results on the stimulation of proliferation of B cells in mice with phosphorothioate-modified ribonucleotides according to the invention (CpG RNA 1668, CpG RNA 1826 and RNA 1982) in comparison with DNA species (with a CpG motif: CpG DNA 1668 and CpG DNA 1826; without a CpG motif: DNA 1982). Medium by itself without a nucleic acid sample serves as the control. ODN with a CpG motif lead to a very high B cell proliferation with almost 90% of proliferating B cells. The ODN DNA 1982 (without a CpG motif), which proved to be inactive in respect of DC stimulation (cf. FIGS. 1 to 5) also caused a moderate B cell proliferation (almost 20% of proliferating cells). In contrast, stimulation of the B cells by the chemically modified oligoribonucleotides according to the invention led to a percentage content of proliferating B cells in the region of or even below that of the negative control (in each case <10% of proliferating cells).

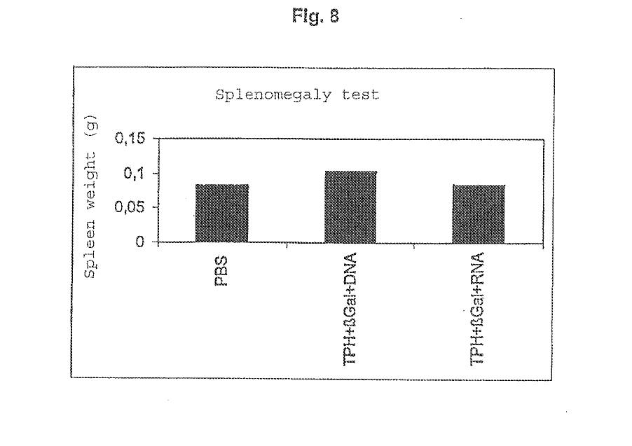

FIG. 8 shows results of an in vivo investigation of the effect of chemically modified RNA according to the invention compared with DNA on the spleen of mice. These were injected subcutaneously with the particular nucleic acid species together with an antigen mixture (peptide TPHARGL (“TPH”)+β-galactosidase (“β-Gal”). After 10 days the spleens were removed from the mice and weighed. The spleen weight is plotted in g on the y axis. The bars in each case show the mean of two independent experiments. While the spleen weight in the mice treated with chemically modified RNA according to the invention+antigen mixture is unchanged compared with the control (PBS) at about 0.08 g, in mice which were injected with DNA+antigen mixture a pronounced splenomegaly is found, which manifests itself in an average weight of the spleen of more than 0.1 g.

The following examples explain the present invention in more detail without limiting it.

### EXAMPLES

The following materials and methods were used to carry out the following examples:

### 1. Cell Culture

Dendritic cells (DC) were obtained by flushing out the rear leg bone marrow of BLAB/c, B6 or MyD88 knock-out mice with medium, treatment with Gey's solution (for lysis of the red blood cells) and filtration through a cell sieve. The cells were then cultured for 6 days in IMDM, containing 10% heat-inactivated foetal calf serum (FCS; from PAN), 2 mM L-glutamine (from Bio Whittaker), 10 mg/ml streptomycin, 10 U/mm penicillin (PEN-STREP, from Bio Whittaker) and 51 U/ml GM-CFS (called “complete medium” in the following), in culture plates having 24 wells. After two and four days, the medium was in each case removed and an equivalent volume of fresh medium which contained the concentration of GM-CFS stated above was added.

### 2. Activation of the DC

After 6 days, the DC were transferred into a culture plate having 96 wells, 200,000 cells in 200 μl complete medium being added to each well. The nucleic acid samples (DNA, chemically modified RNA, mRNA or protamine-stabilized RNA) were added at a concentration of 10 μg/ml.

### 3. RNA Degradation Conditions

In each case 5 μl of the corresponding nucleic acid samples (2 μg/μl DNA, non-modified RNA or chemically modified RNA according to the invention) were incubated in 500 μl complete medium for 2, 26 or 72 h or 15, 30, 45 or 60 min at 37° C. A non-incubated sample (t=0) served as the control. DC were than stimulated with the samples as described under the above point 2.

### 4. Cytokine ELISA

17 hours after addition of the particular stimulant, 100 μl of the supernatant were removed and 100 μl of fresh medium were added. ELISA plates (Nunc Maxisorb) were coated overnight with capture antibodies (Pharmingen) in binding buffer (0.02% NaN₃, 15 mM Na₂CO₃, 15 mM NaHCO₃, pH 9.7). Non-specific binding sites were saturated with phosphate-buffered saline solution (PBS) containing 1% bovine serum albumin (BSA). Thereafter, in each case 100 μl of the particular cell culture supernatant were introduced into a well treated in this way and incubated for 4 hours at 37° C. After 4 washing steps with PBS containing 0.05% een-20, biotinylated antibody was added. The detection reaction was started by addition of streptavidin-coupled radish peroxidase (HRP-streptavidin) and the substrate ABTS (measurement of the absorption at 405 nm).

### 5. Flow Cytometry

For the one-colour flow cytometry, 2×10⁵ cells were incubated for 20 minutes at 4° C. in PBS containing 10% FCS with FITC-conjugated, monoclonal anti-CD86 antibody (Becton Dickinson) in a suitable concentration. After washing twice and fixing in 1% formaldehyde, the cells were analysed with a FACScalibur flow cytometer (Becton Dickinson) and the CellQuestPro software.

### 6. Alloreaction Test by ⁵¹Cr Release

Spleen cells from B6 mice (C57bl6, H-2^{d} haplotype) were incubated with the DC, stimulated according to the above point 2., of BLAB/c mice (H-2^{d} haplotype) in a ratio of 1:3 for 5 days and used as effector cells.

In each case 5,000 EL-4 cells (as a control) or P815 cells (as target cells) were cultured in plates with 96 wells in IMDM with 10% FCS and loaded with ⁵¹Cr for one hour. The ⁵¹Cr-labelled cells were incubated with the effector cells for 5 hours (final volume 200 μl). In each case 3 different ratios of effector or control cells to target cells (E/T) were investigated: E/T=41, 9 or 2. To determine the specific destruction, 50 μl of the supernatant were removed and the radioactivity was measured using a solid phase scintillation plate (Luma Plate-96, Packard) and a scintillation counter for microtitre plates (1450 Microbeta Plus). The percentage content of the ⁵¹Cr release was determined from the amount of ⁵¹Cr released into the medium (A) and compared with the spontaneous ⁵¹Cr release from target cells (B) and the total ⁵¹Cr content of target cells (C), which were lysed with 1% Triton-X-100, the specific destruction resulting from the following formula: % destruction=[(A−B)/(C−B)]×100.

### 7. B Cell Proliferation Test

Fresh spleen cells from a mouse were washed twice with 10 ml PBS and taken up in PBS in a concentration of 1×10⁷ cells/ml. CSFE (FITC-labelled) was added in a final concentration of 500 nM and the mixture was incubated for 3 minutes. It was then washed twice with medium. In each case a non coloured and a coloured sample were analysed in the flow cytometer (FACScalibur; Becton Dickinson). CpG DNA or RNA was added in a concentration of 10 μg/ml to 200,000 cells/well of a culture plate with 96 wells (U-shaped base) in 200 μl of medium. On day 4 after the stimulation, the cells were stained with B220 CyChrome and CD 69 PE and analysed in the FACS.

### 8. In Vivo Investigation of Splenomegaly

50 μg of chemically modified RNA or comparison ODN were injected subcutaneously with an antigen mixture (100 μg peptide TPHARGL+100 μg β-galactosidase) in each case in 200 μl PBS into BALB/c mice (two mice were used for each sample). After 10 days the spleens of the mice were removed and weighed.

### 9. Sequences of the Nucleic Acids Used

Oligodeoxyribonucleotides (ODN):
CpG DNA 1668: 5′-TCCATGACGTTCCTGATGCT-3′
CpG DNA 1826: 5′-TCCATGACGTTCCTGACGTT-3′
DNA 1982: 5′-TCCAGGACTTCTCTCAGGTT-3′
Oligoribonucleotides (phosphorothioate-modified):
CpG RNA 1668: 5′-UCCAUGACGUUCCUGAUGCU-3′
CpG RNA 1826: 5′-UCCAUGACGUUCCUGACGUU-3′
RNA 1982: 5′-UCCAGGACUUCUCUCAGGUU-3′

### Example 1

In order to determine the ability of various nucleic acid species to stimulate maturation of DC, DC were obtained from BALB/c mice and treated with the oligonucleotides described under the above point 6. β-Galactosidase mRNA and pp65 RNA, in each case stabilized by means of protamine, were used as further samples. The release of IL-12 and IL-6 by the stimulated DC was determined by means of ELISA. Stimulation of DC by means of protamine-associated mRNA resulted in a weak release of interleukin. In contrast, the interleukin release caused by the phosphorothioate-modified RNA species according to the invention was considerably greater and was even comparable to the positive control (stimulation by LPS) (FIGS. 1A and 1B). The comparison ODN, which contained a CpG motif, showed an expected release of interleukin by the DC, but the interleukin release was significantly lower compared with the value which was effected by the RNA species of corresponding sequence according to the invention (FIGS. 1A and 1B).

To confirm the induction of the maturation of the DC demonstrated by means of cytokine ELISA, the expression of a specific surface marker for mature DC (CD86) was determined by means of flow cytometry. Phosphorothioate-modified RNA species according to the invention, but not mRNA or protamine-associated mRNA, were able to bring about a significant CD86 expression (FIG. 2).

### Example 2

It was furthermore investigated whether the DC activated by the chemically modified RNA species having an immunostimulating action are capable of causing an immune response in an allogenic system (FIG. 3). For this, mouse spleen cells (B6) were activated with the stimulated DC and brought together, as effector cells, with allogenic target cells (P815), the destruction of the target cells being determined with the aid of a ⁵¹Cr release test. In each case three different dilutions of effector cells were brought into contact with a constant number of target cells here. Phosphorothioate-modified RNA is accordingly very much more capable of causing the maturation of DC to activated cells which can start an immune response by effector cells compared with protamine-stabilized mRNA. Surprisingly, it is to be found here that DC activated by phosphorothioate RNA can induce an immune response which is just as strong as that induced by ODN which have CpG motifs.

### Example 3

It is known that the activation of DC by CpG DN is mediated via TLR-9 (toll-like receptor 9) (Kaisho et al., Trends Immunol. 2001, 22(2): 78-83). It was therefore investigated whether the TLR signal cascade is also involved in the DC activation effected by the chemically modified RNA according to the invention having an immunostimulating action. For this, the activation of DC from B6 wild-type mice was compared with that of DC from 56 mice lacking the protein MyD88 again with the aid of the release of IL-12 and IL-6. MyD88 is involved in the TLR-9 signal cascade. The high release of IL-12 and IL-6 from DC of the B6 wild-type mice confirmed the results of Example 1 (cf. FIGS. 4A and B, black bars). In contrast, stimulation of DC from MyD88 knock-out mice with the same samples led to no activation (cf. FIGS. 4A and B, white bars). These results show that MyD88 and therefore the TLR-9 signal cascade are required both for the CpG DNA-mediated DC activation and for the DC activation mediated by chemically modified RNA.

### Example 4

To investigate whether chemically modified RNA according to the invention is subject to a fast degradation and therefore the danger of a persistence in the organism does not exist, oligoribonucleotides according to the invention were incubated under RNA degradation conditions (37° C., untreated medium, i.e. not RNase-free) for 2, 26 or 72 h and only then fed to the stimulation test with DC. Already after incubation for two hours under RNA degradation conditions, activation of the DC was no longer to be observed in the case of the chemically modified RNA according to the invention, as is demonstrated by the absence of the release of IL-12 (FIG. 5A) and IL-6 (FIG. 5B). In contrast, prior incubation of CpG DNA species has no influence on the activity thereof for DC activation. This shows that the chemically modified RNA according to the invention is already degraded after a relatively short time, which avoids persistence in the organism, which can arise with DNA.

However, the chemically modified RNA according to the invention is not degraded so rapidly that it can no longer display its immunostimulating action. To demonstrate this, the above experiment was repeated with a phosphorothioate-modified oligoribonucleotide according to the invention (RNA 1982), but the incubation was carried out under RNA degradation conditions for only 15, 30, 45 and 60 min. As the release of IL-6 by the DC stimulated in this way shows, even after one hour of incubation under RNA degradation conditions, there is a clear activation of DC (FIG. 6).

### Example 5

The induction of a splenomegaly, which is substantially to be attributed to a potent activation of the B cell proliferation, represents a considerable obstacle to the use of CpG DNA as an immunostimulating adjuvant in vaccines (cf. Monteith et al., see above). It was therefore investigated by means of a B cell proliferation test whether the chemically modified RNA according to the invention has an effect on B cell proliferation. In the B cell proliferation test, an expectedly high content of proliferating cells was detected in the case of stimulation with CpG DNA. In contrast, surprisingly, chemically modified RNA according to the invention was completely inactive in this respect (regardless of any CpG motifs present in the sequence) (FIG. 7).

In order to confirm this surprisingly positive property of the chemically modified RNA according to the invention in vivo, a test vaccine comprising a phosphorothioate oligoribonucleotide according to the invention (RNA 1982) and an antigen mixture of a peptide and β-galactosidase was prepared and injected subcutaneously into mice. A corresponding DNA test vaccine which contained the same antigen mixture in combination with a CpG ODN (CpG DNA 1826) served as a comparison. After 10 days, the spleens were removed from the mice and weighed. Compared with the negative control (PBS), a significant increase in the spleen weight resulted in mice treated with the DNA test vaccine. In contrast, no splenomegaly was found in mice treated with the RNA test vaccine according to the invention, since in this case the spleen weight was unchanged compared with the negative control (FIG. 8). These results show that when the chemically modified RNA is used according to the invention as an immunostimulating agent or as an adjuvant in vaccines, no side effects connected with an undesirable B cell proliferation arise.

Summarizing, it is to be said that chemically modified RNA brings about maturation of DC in vitro. The above examples demonstrate that chemically modified RNA, here in the form of short (e.g. 20-mer) synthetic oligoribonucleotides (which are phosphorothioate-modified), activates immature DC and thus causes maturation thereof, as is demonstrated by determination of the specific cytokine release (FIG. 1) and the expression of surface activation markers (FIG. 2). The DC activation caused by the chemically modified RNA is significantly more potent than that caused by a mixture of mRNA and the polycationic compound protamine, which is known to associate with the RNA and to protect it from nucleases in this way. The DC matured by stimulation with chemically modified RNA according to the invention can start an immune response by effector cells, as demonstrated by a ⁵¹Cr release test in an allogenic system (FIG. 3). The DC activation by the chemically modified RNA according to the invention probably takes place via a TLR-mediated signal cascade (FIG. 4).

It is known of bacterial DNA that because of the presence of non-methylated CG motifs, it has an immunostimulating action; cf. U.S. Pat. No. 5,663,153. This property of DNA can be simulated in DNA oligonucleotides which are stabilized by phosphorothioate modification (U.S. Pat. No. 6,239,116). It is known of RNA which is complexed by positively charged proteins that it has an immunostimulating action (Riedl et al., 2002, see above). It has been possible to demonstrate by the present invention that RNA which is chemically modified is a very much more active immunostimulating agent compared with other, for example protamine-complexed, RNA. In contrast to DNA, no CpG motifs are necessary in such chemically modified RNA oligonucleotides. In contrast to the 20-mer ribonucleotides, free phosphorothioate nucleotides (not shown) do not have an immunostimulating action.

However, the chemically modified immunostimulating RNA of the present invention is superior to the immunostimulating DNA in particular in that RNA is degraded faster and in this way removed from the patient's body, which is why the risk of persistence and of the causing of severe side effects is reduced or avoided (FIGS. 5 and 6). Thus, the use of immunostimulating DNA as an adjuvant for vaccine can cause the formation of anti-DNA antibodies and the DNA can persist in the organism, which can cause e.g. hyperactivation of the immune system, which as is known results in splenomegaly in mice (Montheith et al., 1997, see above). The splenomegaly caused by DNA adjuvants is substantially based on stimulation of B cell proliferation, which does not occur with RNA adjuvants according to the invention (FIGS. 7 and 8). Furthermore, DNA can interact with the host genome, and in particular can cause mutations by integration into the host genome. All these high risks can be avoided using the chemically modified RNA for the preparation of immunostimulating agents or vaccines, in particular for inoculation against or for treatment of cancer or infectious diseases, with better or comparable immunostimulation.

1. A vaccine comprising at least one chemically modified single-stranded RNA molecule which contains at least one analogue of a naturally occurring nucleotide and comprises 8 to 200 nucleotides, wherein the chemically modified single-stranded RNA molecule is not an mRNA and at least one antigen, wherein the at least one antigen is not bound to the chemically modified single-stranded RNA.

2. A vaccine according to claim 1, wherein the chemically modified single-stranded RNA molecule consists of nucleotide analogues.

3. A vaccine according to claim 1, wherein the analogue is selected from the group consisting of phosphorothioates, phosphoroamidates, peptide nucleotides, methylphosphonates, 7-deazaguanosine, 5-methylcytosine and inosine.

4. A vaccine according to claim 3, wherein the analogue is a phosphorothioate.

5. A vaccine according to claim 1, wherein the chemically modified single-stranded RNA molecule consists of about 15 to 31 nucleotides.

6. A vaccine according to claim 1, wherein the chemically modified single-stranded RNA molecule has the sequence 5′-UCCAUGACGUUCCUGAUGCU-3′ (SEQ ID NO:2), 5′-UCCAGGACUUCUCUCAGGUU-3′ (SEQ ID NO:4) or 5′-UCCAUGACGUUCCUGACGUU-3′ (SEQ ID NO:3).

7. A vaccine according to claim 1, further comprising at least one additional adjuvant.

8. A vaccine according to claim 7, wherein the additional adjuvant is selected from the group consisting of cytokines, lipopeptides and CpG oligonucleotides.

9. A vaccine according to claim 1, wherein the antigen is selected from the group consisting of peptide, polypeptides, cells, cell extracts, polysaccharides, polysaccharide conjugates, lipids, glycolipids and carbohydrates.

10. A vaccine according to claim 9, wherein the peptide antigen or polypeptide antigen is in the form of a nucleic acid which codes for the antigen.

11. A vaccine according to claim 10, wherein the nucleic acid is an mRNA.

12. A vaccine according to claim 11, wherein the mRNA is stabilized and/or translation optimized.

13. A vaccine according to claim 1, wherein the antigen is selected from tumour antigens, viral antigens, bacterial antigens, fungal antigens, and protozoal.

14. A vaccine according to claim 13, wherein the viral, bacterial, fungal or protozoal antigen originates from a secreted protein.

15. A vaccine according to claim 13, wherein the antigen is a polyepitope of tumour antigens, viral antigens, bacterial antigens, fungal antigens, or protozoal antigens.

16. A vaccine consisting of at least one antigen; at least one chemically modified single-stranded RNA molecule which contains at least one analogue of a naturally occurring nucleotide and comprises 8 to 200 nucleotides, wherein the chemically modified single-stranded RNA molecule is not an mRNA; a pharmaceutically acceptable carrier; and at least one adjuvant selected from the group consisting of a cytokine, monokine, interleukin, chemokine, growth factor, aluminum hydroxide, Freund's adjuvant, cationic peptide, protamine, cationic polysaccharide, chitosan, lipopeptide and CpG oligonucleotides.

17. The vaccine of claim 16, wherein the adjuvant is a polycationic peptide.

18. The vaccine of claim 16, wherein the adjuvant is protamine.

19. The vaccine of claim 16, wherein the chemically modified single-stranded RNA molecule consists of nucleotide analogues.

20. A vaccine according to claim 16, wherein the analogue is selected from the group consisting of phosphorothioates, phosphoroamidates, peptide nucleotides, methylphosphonates, 7-deazaguanosine, 5-methylcytosine and inosine.

21. A vaccine according to claim 16, wherein the analogue is a phosphorothioate.

22. A vaccine according to claim 16, wherein the chemically modified single-stranded RNA molecule consists of about 15 to about 31 nucleotides.

23. A vaccine according to claim 16, wherein the chemically modified single-stranded RNA molecule has the sequence 5′-UCCAUGACGUUCCUGAUGCU-3′ (SEQ ID NO:2), 5′-UCCAGGACUUCUCUCAGGUU-3′ (SEQ ID NO:2), 5′-UCCAGGACUUCUCUCAGGUU-3′ (SEQ ID NO:4) or 5′-UCCAUGACGUUCCUGACGUU-3′ (SEQ ID NO:3).

24. A vaccine according to claim 16, wherein the adjuvant is a cytokine.

25. A vaccine according to claim 16, wherein the adjuvant is selected from the group consisting of cytokines, lipopeptides and CpG oligonucleotides.

26. A vaccine according to claim 16, wherein the antigen is selected from the group consisting of peptides, polypeptides, cells, cell extracts, polysaccharides, polysaccharide conjugates, lipids, glycolipids and carbohydrates.

27. A vaccine according to claim 26, wherein the peptide antigen or polypeptide antigen is in the form of a nucleic acid which codes for the antigen.

28. A vaccine according to claim 27, wherein the nucleic acid is an mRNA.

29. A vaccine according to claim 28, wherein the mRNA is stabilized and/or translation-optimized.

30. A vaccine according to claim 16, wherein the antigen is selected from tumour antigens, viral antigens, bacterial antigens, fungal antigens, and protozoal.

31. A vaccine according to claim 30, wherein the viral, bacterial, fungal or protozoal antigen originates from a secreted protein.

32. A vaccine according to claim 16, wherein the antigen is a polyepitope of tumour antigens, viral antigens, bacterial antigens, fungal antigens, or protozoal antigens.
